(19) United States
(12) Patent Application Publication
(10) Pub. No.: US 2022/0312804 A1
(43) Pub. Date: Oct. 6, 2022

(54) METHOD FOR ENHANCING THE BIOAVAILABILITY OF HYDROPHILIC ACTIVE COMPOUNDS IN AN AQUEOUS SOLUTION

(71) Applicant: GENIALIS, Henrichemon (FR)

(72) Inventors: Isabelle Desjardins-Lavisse, Henrichemont (FR); Guillaume Gillet, Salbris (FR)

(21) Appl. No.: 17/627,598

(22) PCT Filed: Jul. 9, 2020
(86) PCT No.: PCT/FR2020/051242

§ 371 Date: Jan. 14, 2022

(30) Foreign Application Priority Data
Jul. 15, 2019 (FR) 1907938

(51) Int. Cl. A23L 5/30

(52) U.S. Cl. CPC A23L 5/32

(57) ABSTRACT

The present invention relates to the field of enhancing the bioavailability of active compounds. More particularly, it relates to a method for enhancing the bioavailability of hydrophilic active compounds in solution in an aqueous medium. The hydrophilic compounds, the bioavailability of which is enhanced, can be used, for example, in the cosmetic, agri-food, neutraceutical or pharmaceutical fields.

### CROSS-REFERENCE TO RELATED APPLICATIONS

This application is a national phase entry under 35 U.S.C. § 371 of International Patent Application PCT/FR2020/051242, filed Jul. 9, 2020, designating the United States of America and published as International Patent Publication WO 2021/009450 A1 on Jan. 21, 2021, which claims the benefit under Article 8 of the Patent Cooperation Treaty to French Patent Application Serial No. 1907938, filed Jul. 15, 2019.

### TECHNICAL FIELD

It is known to use a treatment for preparing stable emulsions, comprising a lipid phase in an aqueous phase, using piezoelectric transducers.

### BACKGROUND

International Patent Application Publication No. WO2010/149668 describes a method for preparing an oil-in-water emulsion produced in a container in which a piezoelectric transducer operating at high frequencies, especially of greater than 900 kHz, is submerged.

Moreover, International Patent Application Publication No. WO2017037372 proposes using a similar method, using ultrasound at comparable frequencies, in order to vectorize hydrophobic active substances in the form of a phase dispersed in a continuous hydrophilic phase. In particular, it was proposed to increase the bioavailability of coenzyme Q10 using this method.

Although these methods are highly advantageous for vectorizing active ingredients and increasing the bioavailability thereof, they are associated with the production of an emulsion and are therefore only applicable to hydrophobic active ingredients dispersed in a hydrophilic aqueous solution.

Finally, International Patent Application Publication No. WO2011/057340 describes a method for producing edible products originating from plants and/or fungi that does not use an organic solvent to extract the biological active compounds during the first step of the method. This method comprises the steps of i) obtaining an aqueous medium comprising the matter originating from the plant and/or fungus, ii) thermally treating the medium, then iii) treating it with ultrasound at a frequency of between 20 and 24 kHz. This extraction method aims to improve the activity of the extracts obtained by improving the extraction method itself, i.e., by increasing the amount of active ingredient extracted and thus available in the product obtained. It does not aim to improve the bioavailability of the active ingredients, so as to obtain enhanced efficacy for an identical amount of active ingredient.

### BRIEF SUMMARY

Surprisingly, the inventors have demonstrated the fact that a treatment of hydrophilic compounds present in an aqueous solution using ultrasound makes it possible to significantly improve the bioavailability of these compounds.

Thus, the present disclosure relates to a method for improving the bioavailability of hydrophilic active compounds in solution in an aqueous medium, comprising treating the solution containing the hydrophilic compound with ultrasound at a vibrational frequency of greater than or equal to 1 MHz.

The present disclosure also relates to a composition comprising at least one hydrophilic active compound in an aqueous solution, the composition being treated by applying the method of the present disclosure.

The present disclosure also relates to the use of a composition as described herein in the fields of cosmetics, agri-food, nutraceuticals or pharmaceuticals.

Finally, it relates to a method for preparing a composition according to the present disclosure.

The main advantage of the method according to the present disclosure is that it makes it possible to vectorize, and thereby increase the bioavailability of, hydrophilic molecules while they are in solution in an aqueous medium. This property is entirely surprising and highly advantageous.

The method is particularly advantageous because it makes it possible to formulate hydrophilic compounds while increasing the bioavailability thereof, without adding chemical active ingredients. It paves the way for preparing compositions with a simple formula and for which the efficacy is enhanced.

Thus, this effect has been demonstrated in two different situations:
improved growth of microorganisms when they are incubated in a glucose solution treated beforehand with ultrasound; and
enhanced ease with which caffeine in aqueous solution, treated beforehand with ultrasound, diffuses through the various layers of the skin.

### DESCRIPTION OF THE DRAWINGS

FIG. 1 is an illustration of the reconstructed skin model used in Example 2, as described herein below.

FIG. 2 is a graph illustrating kinetics of diffusion of caffeine through the reconstituted samples as described herein.

Reference: caffeine dispersed in water without any other additions; HF: same as reference, plus application of an ultrasound treatment at a frequency of 1.7 MHz; TA: same as reference, plus addition of two surfactants (Tween 80 and Span) during the dispersion; TA&HF: same as TA, plus application of an ultrasound treatment at a frequency of 1.7 MHz.

### DETAILED DESCRIPTION OF THE INVENTION

A first aspect of the present disclosure relates to a method for improving the bioavailability of a hydrophilic active compound in solution in an aqueous medium, comprising treating the solution containing the compound with ultrasound at a vibrational frequency of greater than or equal to 1 MHz.

For the purposes of the present disclosure, “active compound” is intended to mean a functional compound. This is, in particular, any compound that may have a physiological effect on a biological matrix.

For the purposes of the present disclosure, “aqueous medium” is intended to mean a medium based on water, especially a hydrolate.

In a particular embodiment of the present disclosure, the frequency of the ultrasound is between 1 and 3 MHz.

This method makes it possible to formulate hydrophilic compounds in solution in an aqueous medium via a method, without adding chemical active ingredients. It is therefore a very simple formulation that may only contain the hydrophilic active compound, with the vectorization effect being obtained by virtue of the ultrasound treatment.

Without being bound by this theory, it has been considered that ultrasound induces a “capsuleless vectorization” (without a vectorizing agent), probably due to ionic rearrangements around the hydrophilic compounds present in the solution. This concept is entirely novel and differs from the concept of vectorization as used to date, which is based on active enclosing, based on the formation of a capsule containing the active compound, by virtue of the addition of one or more other compounds to the aqueous medium. This is, in particular, the case in oil-in-water emulsions, where each dispersed oil droplet forms a capsule containing one or more hydrophobic active ingredients dispersed in an oil. The formation of the capsule thus requires the addition at least of an oil and, very generally, also of surfactant and/or stabilizer in order to keep the dispersion stable over time.

A second aspect of the present disclosure relates to a composition comprising at least one hydrophilic active compound in solution in an aqueous medium, wherein the composition is treated by applying the method according to the present disclosure.

Such a hydrophilic compound may be, for example, a sugar, a polar amino acid, a flavoring or a pigment, a trace element, an antioxidant, a salicylic analgesic, an antifungal, an antibiotic (of glycopeptide type, for example) or any other water-soluble active substance.

In particular, such a hydrophilic compound may be, for example, caffeine, glucose, vitamin C, vitamin B9 (folic acid), hyaluronic acid, urea, acetylsalicylic acid or vanillin.

The formulation of the composition may be limited to the hydrophilic active compound itself in an aqueous medium. The composition may also be used as an ingredient for the preparation of complex compositions.

A third aspect of the present disclosure relates to the use of a composition according to the present disclosure in the field of cosmetics or consumables.

A cosmetic composition may be a lotion, a cream, or any other formulation comprising an aqueous solution.

In the field of consumables, the compositions according to the present disclosure may be, for example, beverages comprising hydrophilic natural active ingredients chosen for their beneficial properties, or flavorings.

A fourth aspect of the present disclosure relates to the use of a composition according to the present disclosure in the field of pharmaceuticals or nutraceuticals.

The compositions for pharmaceutical use may be, in particular, ocular or injectable preparations for which reducing or eliminating excipients is highly advantageous, in particular, in order to limit side effects.

Indeed, the regular administration of complex ocular compositions may induce allergic reactions or inflammatory responses. Proposing a simple formula without a chemical vectorizing agent limits this risk. Moreover, the dose can be reduced because the bioavailability of the active ingredient is increased.

An injectable composition may be, for example, a vaccine composition or a vaccine composition support in which the antigen or the adjuvants are more bioavailable and therefore more effective for an equivalent dose, thereby making it possible to reduce the dose.

A fifth aspect of the present disclosure relates to a method for preparing a composition as described herein, comprising:
dissolving a hydrophilic active compound in an aqueous solution; and
subjecting this solution to a treatment by ultrasound at a vibrational frequency of greater than or equal to 1 MHz.

The treatment time may vary and is dependent on the volume of preparation to be treated, on the molecule(s) to be vectorized, and on the number, type and power of the ultrasonic transmitter.

The present disclosure will be better understood on reading the following examples, provided by way of illustration and in no event to be considered as limiting the scope of the present disclosure.

### EXAMPLES

### Example 1: Bioavailability of Glucose: Application in the Growth of Microorganisms

a—Materials and Methods

Glucose syrup (78% glucose in water) was diluted in water so as to obtained 3 liters of a preparation containing 10% glucose, optionally adjusted to a pH equal to 7 by adding sodium hydroxide. The preparation was subsequently separated into three batches, each of 1 liter:
a control batch not subjected to any treatment;
a batch treated for 30 min using an ultrasonic emitter generating a wave with a frequency of 1.7 MHz; and
a batch treated for 60 min using an ultrasonic emitter generating a wave with a frequency of 1.7 MHz.

For each of the solutions thus produced, 6 30 ml flasks of the solution are prepared, to which the following are added:
nothing for 2 of them;
50 g of a bacterial preparation mixture for powdered yogurt (Lactobacillus bulgaricus and Streptococcus thermophilus) for two of them; and
50 g of powdered baker's yeast (Saccharomyces cerevisiae).

The different flasks are analyzed by UV-visible spectroscopy in order to measure absorbance at 600 nm. The analyses are carried out just after the preparation (t0) and 24 h later (t24).

b—Results

The results for absorbance at 600 nm are presented in table 1. They are expressed as the difference between the absorbances obtained at t24 and those obtained at t0, in other words:

Aₜ₂₄−Aₜ₀

TABLE 1
Difference between the absorbance after 24 h and
the initial absorbance, measured at 600 nm.

| | | Lact. bulgaricus | |
|---|---|---|---|
| | Not | and Strep. | Saccharomyces |
| | inoculated | thermophilus | cerevisiae |
| No treatment | 0.0085 | 0.023 | −0.002 |
| Treatment | 0.012 | 0.0335 | 0.1685 |
| 30 min | | | |
| Treatment | 0.013 | 0.045 | 0.128 |
| 60 min | | | |

It is observed that the increase in absorbance is greater for the inoculated products when they have been subjected to the treatment, and that this is particularly significant for the yeast. Although the values are lower for the bacteria, they are also significant; this less pronounced result could be explained by the fact that glucose alone is not sufficient for effective bacterial growth.

The results show an enhanced growth in the case of bacteria and in the case of yeasts when they are incubated in a glucose-based medium that has been treated beforehand with ultrasound. This observation confirms better bioavailability of the glucose after ultrasound treatment.

### Example 2: Bioavailability of Caffeine: Cutaneous Application

a—Materials and Methods

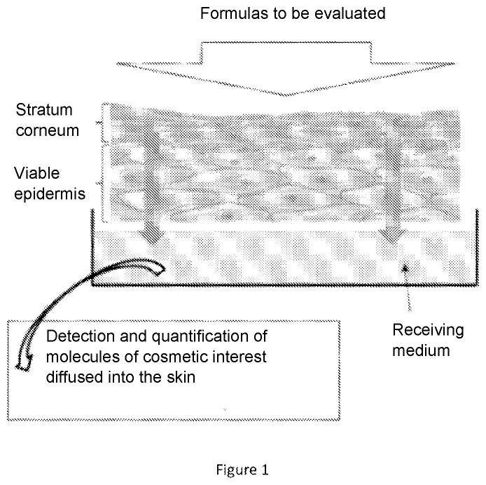

12-well plates each containing an EPISKIN reconstructed skin sample are used. The system used is shown in FIG. 1. The formulas to be evaluated are deposited on the surface. The receiving medium in which the skin samples are immersed is recovered and replaced every 30 min up to 3 h, then every hour up to 12 h. The recovered fraction is analyzed by HPLC (High Performance Liquid Chromatography) in order to quantify the caffeine present therein. The total amount of caffeine that has migrated through the skin sample is thus obtained by accumulating the amounts measured each time a sample is taken.

The HPLC is carried out by a Dionex Ultimate 3000 system, using an Interchim Vintage Series KRC18 separating column. The analyses are carried out on an injected volume of 10 μl in a mobile phase comprising a 50/50 (w/w) mixture of methanol and ultrapure water circulating at a flow rate of 1 ml/min.

The compositions comprising caffeine in solution in water are all prepared so as to ultimately contain 2% caffeine and the pH thereof is optionally adjusted to a pH equal to 7 by adding sodium hydroxide. The preparations evaluated during this experiment are as follows:
reference: composition prepared without surfactant and without treatment;
hydrofeeling (or HF): composition prepared without surfactant, with a treatment by ultrasound at a vibrational frequency of 1.7 MHz for 30 min;
TA: composition prepared with surfactants (3% Tween80 and 2% Span in the final preparation), without ultrasound treatment; and
TA&HF: composition prepared with surfactants (3% Tween80 and 2% Span in the final preparation), with a treatment by ultrasound at a vibrational frequency of 1.7 MHz for 30 min.

b—Results

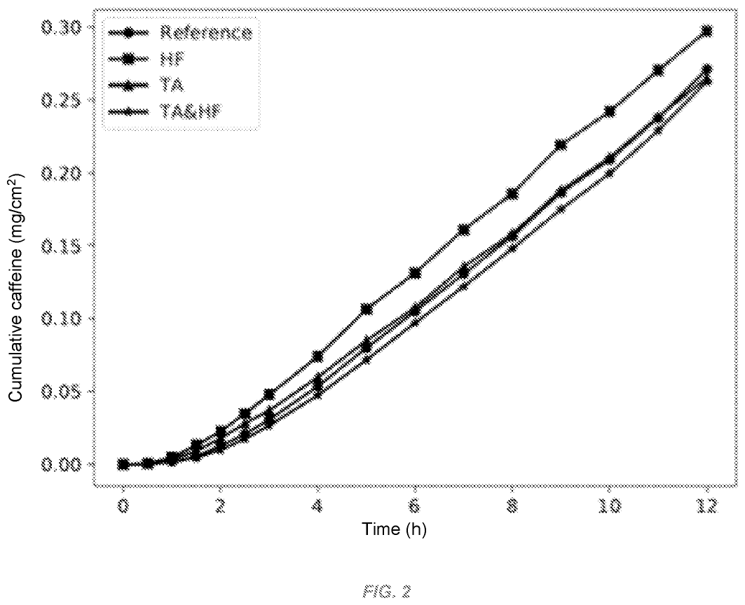

The results are presented in FIG. 2.

FIG. 2 shows that all the kinetics of diffusion of the caffeine are comparable with the exception of that denoted HF, corresponding to the treatment according to the present disclosure. This increased rate is observed above all at the start of the diffusion, with the plots being relatively parallel once the constant flow has been established. This may be reflected by the observation of two parameters calculated from the above plots: the constant flow J_{c} and the time lag t_{lag}.

J_{c} is calculated as the gradient of the line observed in the second part of the plot. t_{lag} is defined as the time, on the x axis, separating the “straight line” part of the plot, corresponding to the constant flow, and the “curved” part of the plot, corresponding to the acceleration of the flow rate at the start of diffusion. This data is presented for each plot in Table 2 below:

TABLE 2
Flow (J_{c}) and time lag (t_{lag}) calculated for the
vectorized caffeine diffusions in FIG. 2 (the names
of the preparations are the same as in the figure).

| Preparation | J_{c}(μg · cm⁻² · h⁻¹) | t_{lag} (h) |
|---|---|---|
| Reference | 26 ± 0.5 | 1.9 ± 0.08 |
| HF | 28 ± 1.8 | 1.2 ± 0.06 |
| TA | 26 ± 1.4 | 1.7 ± 0.07 |
| TA&HF | 26 ± 1.4 | 2.2 ± 0.04 |

The calculated data presented in table 2 confirms the previous observations. The constant flow, once reached, is the same regardless of the conditions of preparation of the caffeine, which reflects the fact that the actual structure of the skin samples and the layers constituting same are not adversely affected. In particular, the treatment according to the present disclosure does not induce any denaturing of the skin samples.

However, the time lag is very different between the kinetics obtained by virtue of the product prepared according to the present disclosure, on the one hand, and all the others, on the other. This difference confirms that the treatment according to the present disclosure causes the ease with which the hydrophilic active substance diffuses through the different layers of the skin. Enhanced bioavailability can be deduced therefrom, since the ease with which it reaches the target cells (located in the hypodermis in the case of caffeine) has been improved.

1. A method for improving the bioavailability of a hydrophilic active compound in solution in an aqueous medium, comprising treating the solution containing the compound with ultrasound at a vibrational frequency of greater than or equal to 1 MHz.

2. A composition comprising at least one hydrophilic active compound in solution in an aqueous medium, wherein the composition is treated by applying the method as defined in claim 1.

3. The composition according to claim 2, wherein the hydrophilic active compound is selected from sugars, polar amino acids, flavorings or water-soluble pigments, trace elements, antioxidants, salicylic analgesics, antifungals, antibiotics or any other water-soluble active substances.

4. The composition according to claim 3, wherein the hydrophilic active compound is selected from glucose, caffeine, vitamin C, vitamin B9 (folic acid), hyaluronic acid, urea, acetylsalicylic acid or vanillin.

5. A method comprising using a composition as defined in claim 2 in the field of cosmetics or consumables.

6. The method according to claim 5, wherein the composition is a beverage comprising hydrophilic natural active substances.

7. The composition according to claim 2, for use thereof in the field of pharmaceuticals or nutraceuticals.

8. The composition according to claim 7, wherein the composition is as an injectable composition or a support for an injectable composition.

9. The composition according to claim 7, wherein the composition is an ocular composition or a support for an ocular composition.

10. A method for preparing a composition comprising at least one hydrophilic active compound in solution in an aqueous medium, comprising:
dissolving the at least one hydrophilic active compound in an aqueous solution; and
subjecting the aqueous solution to a treatment by ultrasound at a vibrational frequency of greater than or equal to 1 MHz.
